SEQUENCE LISTING (52 sequences)

SEQ ID NO: 1 - DNA, 23 bp, synthetic construct
  source 1..23: mol_type = other DNA; note = Primer PR-6 F
agctagaccctaaatccttgtgt

SEQ ID NO: 2 - DNA, 20 bp, synthetic construct
  source 1..20: mol_type = other DNA; note = Primer PR-6 R
actcctctcacgtcttcagc

SEQ ID NO: 3 - DNA, 20 bp, synthetic construct
  source 1..20: mol_type = other DNA; note = Primer PR-7 F
tcctacacatgtgcttggaa

SEQ ID NO: 4 - DNA, 20 bp, synthetic construct
  source 1..20: mol_type = other DNA; note = Primer PR-7 R
atccatgccctaatgcctcc

SEQ ID NO: 5 - DNA, 23 bp, synthetic construct
  source 1..23: mol_type = other DNA; note = Primer PR-8 F
acttggggaaatctcaaaagtct

SEQ ID NO: 6 - DNA, 22 bp, synthetic construct
  source 1..22: mol_type = other DNA; note = Primer PR-8 R
ggcaaaagccttagagaaatgc

SEQ ID NO: 7 - DNA, 20 bp, synthetic construct
  source 1..20: mol_type = other DNA; note = Primer PR-10 F
atctatggggtcgcacagag

SEQ ID NO: 8 - DNA, 21 bp, synthetic construct
  source 1..21: mol_type = other DNA; note = Primer PR-10 R
accagagttcagaaccagtgt

SEQ ID NO: 9 - DNA, 21 bp, synthetic construct
  source 1..21: mol_type = other DNA; note = Primer PR-12 F
tccccattaccacacctaagt

SEQ ID NO: 10 - DNA, 20 bp, synthetic construct
  source 1..20: mol_type = other DNA; note = Primer PR-12 R
acatccaggagagtgtcagc

SEQ ID NO: 11 - DNA, 20 bp, synthetic construct
  source 1..20: mol_type = other DNA; note = Primer PR-14 F
agatgaactgctctgggaag

SEQ ID NO: 12 - DNA, 21 bp, synthetic construct
  source 1..21: mol_type = other DNA; note = Primer PR-14 R
aatgggaaagtatgaaaggca

SEQ ID NO: 13 - DNA, 20 bp, synthetic construct
  source 1..20: mol_type = other DNA; note = Primer PR-16 F
gtttctgccctcaatctgtt

SEQ ID NO: 14 - DNA, 23 bp, synthetic construct
  source 1..23: mol_type = other DNA; note = Primer PR-16 R
agacttttacatctcagctaagc

SEQ ID NO: 15 - DNA, 21 bp, synthetic construct
  source 1..21: mol_type = other DNA; note = Primer PR-22 F
tcagatctccctcaatgcagg

SEQ ID NO: 16 - DNA, 23 bp, synthetic construct
  source 1..23: mol_type = other DNA; note = Primer PR-22 R
gtgttagtttcaggtgtagaaca

SEQ ID NO: 17 - DNA, 20 bp, synthetic construct
  source 1..20: mol_type = other DNA; note = Primer PR-25 F
ccaagagtgaagttgtgcca

SEQ ID NO: 18 - DNA, 20 bp, synthetic construct
  source 1..20: mol_type = other DNA; note = Primer PR-25 R
aaccaagtgctctgtttcag

SEQ ID NO: 19 - DNA, 20 bp, synthetic construct
  source 1..20: mol_type = other DNA; note = Primer PR-26 F
aaactggaatgggttgccac

SEQ ID NO: 20 - DNA, 23 bp, synthetic construct
  source 1..23: mol_type = other DNA; note = Primer PR-26 R
agggatgatgtattagttagggt

SEQ ID NO: 21 - DNA, 20 bp, synthetic construct
  source 1..20: mol_type = other DNA; note = Primer PR-28 F
acccaatttcgagcttccag

SEQ ID NO: 22 - DNA, 22 bp, synthetic construct
  source 1..22: mol_type = other DNA; note = Primer PR-28 R
tgtactagttttccagagctgc

SEQ ID NO: 23 - DNA, 20 bp, synthetic construct
  source 1..20: mol_type = other DNA; note = Primer PR-30 F
attctctcgggccatctgtg

SEQ ID NO: 24 - DNA, 20 bp, synthetic construct
  source 1..20: mol_type = other DNA; note = Primer PR-30 R
cactgctggaatggtcaagg

SEQ ID NO: 25 - DNA, 20 bp, synthetic construct
  source 1..20: mol_type = other DNA; note = Primer PR-40 F
ccgttctccaggggaatctt

SEQ ID NO: 26 - DNA, 20 bp, synthetic construct
  source 1..20: mol_type = other DNA; note = Primer PR-40 R
ttcacagggccttcagaaac

SEQ ID NO: 27 - DNA, 20 bp, synthetic construct
  source 1..20: mol_type = other DNA; note = Primer PR-42 F
tagagcccaatccaaggtcc

SEQ ID NO: 28 - DNA, 21 bp, synthetic construct
  source 1..21: mol_type = other DNA; note = Primer PR-42 R
tgcagagccatcagttaagac

SEQ ID NO: 29 - DNA, 19 bp, synthetic construct
  source 1..19: mol_type = other DNA; note = Primer PR-46 F
ctctgtcctccgctgtctc

SEQ ID NO: 30 - DNA, 20 bp, synthetic construct
  source 1..20: mol_type = other DNA; note = Primer PR-46 R
caaattgccttgtccctgcc

SEQ ID NO: 31 - DNA, 22 bp, synthetic construct
  source 1..22: mol_type = other DNA; note = Primer PR-53 F
tccctgatctgtaacaaatgtg

SEQ ID NO: 32 - DNA, 20 bp, synthetic construct
  source 1..20: mol_type = other DNA; note = Primer PR-53 R
ccttagcgtcacacacacag

SEQ ID NO: 33 - DNA, 20 bp, synthetic construct
  source 1..20: mol_type = other DNA; note = Primer PR-58 F
cagagtcggtcatgatggga

SEQ ID NO: 34 - DNA, 20 bp, synthetic construct
  source 1..20: mol_type = other DNA; note = Primer PR-58 R
tgcttccgaccaagtgtttg

SEQ ID NO: 35 - DNA, 21 bp, synthetic construct
  source 1..21: mol_type = other DNA; note = Primer PR-63 F
ggggcatcacaattccattct

SEQ ID NO: 36 - DNA, 23 bp, synthetic construct
  source 1..23: mol_type = other DNA; note = Primer PR-63 R
tccaactttcagaccataaatcc

SEQ ID NO: 37 - DNA, 22 bp, synthetic construct
  source 1..22: mol_type = other DNA; note = Primer PR-64 F
ccctctctgttgtagttgttgg

SEQ ID NO: 38 - DNA, 20 bp, synthetic construct
  source 1..20: mol_type = other DNA; note = Primer PR-64 R
acaatcctgcaaaagccctc

SEQ ID NO: 39 - DNA, 20 bp, synthetic construct
  source 1..20: mol_type = other DNA; note = Primer PR-65 F
ggcaagtgaggtgatgaagc

SEQ ID NO: 40 - DNA, 22 bp, synthetic construct
  source 1..22: mol_type = other DNA; note = Primer PR-65 R
tggccttctagagttgttttgc

SEQ ID NO: 41 - DNA, 22 bp, synthetic construct
  source 1..22: mol_type = other DNA; note = Primer PR-69 F
agcaatgaagaccagcataacc

SEQ ID NO: 42 - DNA, 21 bp, synthetic construct
  source 1..21: mol_type = other DNA; note = Primer PR-69 R
cccatcaatctttgcagtccc

SEQ ID NO: 43 - DNA, 20 bp, synthetic construct
  source 1..20: mol_type = other DNA; note = Primer PR-71 F
tttgctgcctcgacaaacag

SEQ ID NO: 44 - DNA, 21 bp, synthetic construct
  source 1..21: mol_type = other DNA; note = Primer PR-71 R
tcccgcattacaagcagattc

SEQ ID NO: 45 - DNA, 20 bp, synthetic construct
  source 1..20: mol_type = other DNA; note = Primer PR-72 F
tggttctgtacaagcttcag

SEQ ID NO: 46 - DNA, 20 bp, synthetic construct
  source 1..20: mol_type = other DNA; note = Primer PR-72 R
cagtctggattgaggggtct

SEQ ID NO: 47 - DNA, 23 bp, synthetic construct
  source 1..23: mol_type = other DNA; note = Primer PR-85 F
gttccaagctaagcatttaaggc

SEQ ID NO: 48 - DNA, 20 bp, synthetic construct
  source 1..20: mol_type = other DNA; note = Primer PR-85 R
gggaacacatgtacacccgt

SEQ ID NO: 49 - DNA, 20 bp, synthetic construct
  source 1..20: mol_type = other DNA; note = Primer PR-86 F
cgctttaccatctcagccac

SEQ ID NO: 50 - DNA, 20 bp, synthetic construct
  source 1..20: mol_type = other DNA; note = Primer PR-86 R
agtcatccatcggtccatgt

SEQ ID NO: 51 - DNA, 21 bp, synthetic construct
  source 1..21: mol_type = other DNA; note = Primer PR-97 F
tgatcacagaaccccacaagt

SEQ ID NO: 52 - DNA, 20 bp, synthetic construct
  source 1..20: mol_type = other DNA; note = Primer PR-97 R
taggggaagcctgttaagca

(19) United States
(12) Patent Application Publication
(10) Pub. No.: US 2026/0071274 A1
(43) Pub. Date: Mar. 12, 2026

(54) COMBINATION OF SIMPLE SEQUENCE REPEATS (SSR) MOLECULAR MARKER, PRIMER COMBINATION, KIT FOR PROCAPRA PRZEWALSKII, AND USE

(71) Applicant: NORTHWEST INSTITUTE OF PLATEAU BIOLOGY, CHINESE ACADEMY OF SCIENCES, Xining (CN)

(72) Inventors: Zhenyuan Cai, Xining (CN); Ning Chang, Xining (CN); Tongzuo Zhang, Xining (CN); Pengfei Song, Xining (CN); Jingjie Zhang, Xining (CN); Daoxin Liu, Xining (CN)

(21) Appl. No.: 18/553,402

(22) PCT Filed: Jun. 27, 2023
(86) PCT No.: PCT/CN2023/102551

§ 371 Date: Sep. 29, 2023

(30) Foreign Application Priority Data
May 29, 2023 (CN) 202310615110.8

(51) Int. Cl. C12Q 1/6876

(52) U.S. Cl. CPC C12Q 1/6876; C12Q 2600/156

(57) ABSTRACT

The present disclosure provides a combination of single sequence repeat (SSR) molecular marker for Procapra przewalskii, a primer combination, a kit, and use, and relates to the technical field of molecular biology. In the present disclosure, design of SSR primers and screening of SSR loci are conducted based on a whole-genome sequencing data of Procapra przewalskii. 26 pairs of obtained SSR primers can stably amplify a target product, and are highly polymorphic, and can be used for population genetic diversity detection, population genetic structure analysis, evolution, and kinship research of the Procapra przewalskii. A combination of polymorphic SSR molecular marker of Procapra przewalskii selected can be used for individual identification of the Procapra przewalskii with a high accuracy. The experimental results show that the combination of SSR molecular marker of Procapra przewalskii can meet the needs of individual identification.

### CROSS REFERENCE TO RELATED APPLICATION

This application is a national stage application of International Patent Application No. PCT/CN2023/102551, filed on Jun. 27, 2023, which claims the priority to Chinese Patent Application 202310615110.8, titled “COMBINATION OF SIMPLE SEQUENCE REPEATS (SSR) MOLECULAR MARKER, PRIMER COMBINATION, KIT FOR PROCAPRA PRZEWALSKII, AND USE”, filed with the China National Intellectual Property Administration (CNIPA) on May 29, 2023, both of which are incorporated herein by reference in their entireties.

### REFERENCE TO SEQUENCE LISTING

A computer readable XML file entitled “HLPCTP20230604735”, created on Sep. 14, 2023 with a file size of about 56,094 bytes, contains the sequence listing for this application, has been filed with this application, and is hereby incorporated by reference in its entirety.

### TECHNICAL FIELD

The present disclosure belongs to the technical field of molecular biology, and in particular relates to a combination of simple sequence repeats (SSR) molecular marker, a primer combination, a kit for Procapra przewalskii, and use.

### BACKGROUND

Procapra przewalskii, belonging to Procapra, Bovidae, Artiodactyla, is endemic animal of the Qinghai-Tibet Plateau, only distributed in the area around Qinghai Lake now, which is a flagship species in the Qinghai Lake Basin, listed in the first order of key protected wild animal list in China. With the implementation of various protection measures and the strengthening of protection efforts, the population of Procapra przewalskii has recovered rapidly. In May 2022, the released data from Qinghai Province Forestry and Grassland Bureau showed that there were more than 2,800 adult individuals of Procapra przewalskii in the wild.

Simple sequence repeats (SSR), also known as microsatellite sequences, is a tandem repeat sequence generated by multiple repetitions of 1 to 6 nucleotides that serve as a repeating unit. The SSR exists almost in the whole genome, and is polymorphic due to the difference in repeat units and repeat numbers between different alleles.

At present, many scholars have conducted a lot of researches on the population distribution, population size, habitat selection, feeding strategies, feeding habits, and threatened factors of the Procapra przewalskii. Some scholars have also conducted researches on the population genetic diversity of the Procapra przewalskii using mitochondrial molecular markers. However, there is no relevant report in the prior art on individual identification of the Procapra przewalskii using SSR markers.

### SUMMARY

In view of this, an objective of the present disclosure is to provide a combination of 4-base single sequence repeat (SSR) molecular marker, a primer combination, a kit for Procapra przewalskii, and use. In the present disclosure, polymorphic SSR markers are developed based on a whole-genome sequencing data of Procapra przewalskii, thereby satisfying the requirements for individual identification of Procapra przewalskii.

To achieve the above objective, the present disclosure provides the following technical solutions:

The present disclosure provides an combination of SSR molecular marker for Procapra przewalskii, the combination of SSR molecular marker including one or more of PR-6, PR-7, PR-8, PR-10, PR-12, PR-14, PR-16, PR-22, PR-25, PR-26, PR-28, PR-30, PR-40, PR-42, PR-46, PR-53, PR-58, PR-63, PR-64, PR-65, PR-69, PR-71, PR-72, PR-85, PR-86, and PR-97; where the SSR molecular markers are sequentially amplified by following primer pairs, the primer pairs have sequences set forth in SEQ ID NO: 1-2, SEQ ID NO: 3-4, SEQ ID NO: 5-6, SEQ ID NO: 7-8, SEQ ID NO: 9-10, SEQ ID NO: 11-12, SEQ ID NO: 13-14, SEQ ID NO: 15-16, SEQ ID NO: 17-18, SEQ ID NO: 19-20, SEQ ID NO: 21-22, SEQ ID NO: 23-24, SEQ ID NO: 25-26, SEQ ID NO: 27-28, SEQ ID NO: 29-30, SEQ ID NO: 31-32, SEQ ID NO: 33-34, SEQ ID NO: 35-36, SEQ ID NO: 37-38, SEQ ID NO: 39-40, SEQ ID NO: 41-42, SEQ NO: 43-44, SEQ ID NO: 45-46, SEQ ID NO: 47-48, SEQ ID NO: 49-50, SEQ ID NO: 51-52.

Preferably, the combination of SSR molecular marker includes the PR-6, the PR-8, the PR-12, the PR-22, the PR-25, the PR-30, the PR-40, the PR-58, the PR-63, the PR-69, the PR-71, the PR-72, the PR-85, the PR-86, and the PR-97.

The present disclosure further provides an primer combination of SSR molecular marker for Procapra przewalskii, including any one or more of the following primer pairs, where the primer pairs have sequences set forth in SEQ ID NO: 1-2, SEQ ID NO: 3-4, SEQ ID NO: 5-6, SEQ ID NO: 7-8. SEQ ID NO: 9-10, SEQ ID NO: 11-12, SEQ ID NO: 13-14, SEQ ID NO: 15-16, SEQ ID NO: 17-18, SEQ ID NO: 19-20, SEQ ID NO: 21-22, SEQ ID NO: 23-24, SEQ ID NO: 25-26, SEQ ID NO: 27-28, SEQ ID NO: 29-30, SEQ ID NO: 31-32, SEQ ID NO: 33-34, SEQ ID NO: 35-36, SEQ ID NO: 37-38, SEQ ID NO: 39-40, SEQ ID NO: 41-42, SEQ NO: 43-44, SEQ ID NO: 45-46, SEQ ID NO: 47-48. SEQ ID NO: 49-50, SEQ ID NO: 51-52.

The present disclosure further provides a kit for identifying an individual of Procapra przewalskii, including the primer combination of SSR molecular marker.

Preferably, the kit further includes a genome extraction reagent and a PCR reaction reagent.

Preferably, the kit further includes a reagent for capillary electrophoresis.

The present disclosure further provides use of the primer combination of SSR molecular marker or the kit in identification and analysis for an individual of Procapra przewalskii.

The present disclosure further provides use of the primer combination of SSR molecular marker or the kit in detection for a population genetic diversity of Procapra przewalskii.

The present disclosure further provides a method for individual identification of Procapra przewalskii, including the following steps: extracting genomic DNAs from samples of the Procapra przewalskii, subjecting the genomic DNA to PCR amplification with the primer pairs, conducting capillary electrophoresis, determining genotypes at loci of SSR molecular markers, and conducting the individual identification; and when the genotypes at loci of the SSR molecular markers are the same or only one genotype at one locus is different, determining that the samples are come from the same individual.

Preferably, the sample is selected from the group consisting of a feces and a tissue.

Compared with the prior art, the present disclosure has the following beneficial effects:

In the present disclosure, design of SSR primers and screening of SSR loci are conducted based on a whole-genome sequencing data of Procapra przewalskii. 26 pairs of obtained SSR primers can stably amplify a target product, and are highly polymorphic. Therefore, the SSR primers can be used for population genetic diversity detection, population genetic structure analysis, evolution, and kinship research of the Procapra przewalskii. A combination of polymorphic SSR molecular marker of Procapra przewalskii selected can be used for individual identification of the Procapra przewalskii with a high accuracy.

The experimental results show that the combination of SSR molecular marker of Procapra przewalskii can meet the needs of individual identification, where conducting individual identification on 33 feces samples of Procapra przewalskii subadults shows that these feces samples come from 24 different individuals which is consistent with the number of Procapra przewalskii subadults in Jiangxigou Procapra przewalskii Rescue Center.

### BRIEF DESCRIPTION OF THE DRAWINGS

FIG. 1 shows 33 feces samples from Procapra przewalskii subadults;

FIG. 2 shows an agarose gel electrophoresis result of a total genomic DNA of a part of the feces samples from Procapra przewalskii; where the leftmost part is a 100 bp DNA Ladder (Dye Plus) (TaKaRa), and 1 to 15 represent DNA samples of Procapra przewalskii;

FIGS. 3A-B show allele frequencies of 26 SSR loci in 15 Procapra przewalskii samples;

FIG. 4 shows a probability of identity of 15 SSR loci selected for individual identification; and

FIGS. 5A-D show capillary electrophoresis results of SSR loci in samples 2501 (A), 3201 (B), 3203 (C), and 3306 (D) for individual identification of Procapra przewalskii.

### DETAILED DESCRIPTION OF THE EMBODIMENTS

The present disclosure provides an combination of SSR molecular marker for Procapra przewalskii, the combination of SSR molecular marker including one or more of PR-6, PR-7, PR-8, PR-10, PR-12, PR-14, PR-16, PR-22, PR-25, PR-26, PR-28, PR-30, PR-40, PR-42, PR-46, PR-53, PR-58, PR-63, PR-64, PR-65, PR-69, PR-71, PR-72, PR-85, PR-86, and PR-97; where the SSR molecular markers are sequentially amplified by following primer pairs, the primer pairs have sequences set forth in SEQ ID NO: 1-2, SEQ ID NO: 3-4, SEQ ID NO: 5-6, SEQ ID NO: 7-8, SEQ ID NO: 9-10, SEQ ID NO: 11-12, SEQ ID NO: 13-14, SEQ ID NO: 15-16, SEQ ID NO: 17-18, SEQ ID NO: 19-20, SEQ ID NO: 21-22, SEQ ID NO: 23-24, SEQ ID NO: 25-26, SEQ ID NO: 27-28, SEQ ID NO: 29-30, SEQ ID NO: 31-32, SEQ ID NO: 33-34, SEQ ID NO: 35-36, SEQ ID NO: 37-38, SEQ ID NO: 39-40, SEQ ID NO: 41-42, SEQ NO: 43-44, SEQ ID NO: 45-46, SEQ ID NO: 47-48, SEQ ID NO: 49-50, SEQ ID NO: 51-52.

In the present disclosure, the combination of SSR molecular marker includes preferably the PR-6, the PR-8, the PR-12, the PR-22, the PR-25, the PR-30, the PR-40, the PR-58, the PR-63, the PR-69, the PR-71, the PR-72, the PR-85, the PR-86, and the PR-97. The probability of identity test results show that a combination of the above 15 SSR molecular markers can meet the needs of individual identification.

The present disclosure further provides an primer combination of SSR molecular marker for Procapra przewalskii, including any one or more of the following primer pairs, where the primer pairs have sequences set forth in SEQ ID NO: 1-2, SEQ ID NO: 3-4, SEQ ID NO: 5-6, SEQ ID NO: 7-8, SEQ ID NO: 9-10, SEQ ID NO: 11-12, SEQ ID NO: 13-14, SEQ ID NO: 15-16, SEQ ID NO: 17-18, SEQ ID NO: 19-20, SEQ ID NO: 21-22, SEQ ID NO: 23-24, SEQ ID NO: 25-26, SEQ ID NO: 27-28, SEQ ID NO: 29-30, SEQ ID NO: 31-32, SEQ ID NO: 33-34, SEQ ID NO: 35-36, SEQ ID NO: 37-38, SEQ ID NO: 39-40, SEQ ID NO: 41-42, SEQ NO: 43-44, SEQ ID NO: 45-46, SEQ ID NO: 47-48, SEQ ID NO: 49-50, SEQ ID NO: 51-52.

The greater the number of alleles at each locus, the more polymorphic the population can be. In the present disclosure, a total of 143 alleles are detected in an amplified product of the primer combination of SSR molecular marker for Procapra przewalskii, and the number of alleles ranges from 2 (PR-97) to 10 (PR-14). It is seen that in the present disclosure, a primer combination of the above 26 SSR molecular marker of Procapra przewalskii can stably amplify a target product, and are highly polymorphic, and can be used for population genetic diversity detection, population genetic structure analysis, evolution, and kinship research of the Procapra przewalskii.

The present disclosure further provides a kit for identifying an individual of Procapra przewalskii, including the primer combination of SSR molecular marker.

In the present disclosure, the kit further includes preferably a genome extraction reagent and a PCR reaction reagent.

In the present disclosure, the kit further includes preferably a reagent for capillary electrophoresis.

The present disclosure further provides use of the primer combination of SSR molecular marker or the kit in identification and analysis for an individual of Procapra przewalskii.

In the present disclosure, 15 loci of the combination of SSR molecular marker of Procapra przewalskii can be used for individual identification on 33 feces samples of Procapra przewalskii subadults. The experimental results show that these 33 feces samples of Procapra przewalskii subadults come from 24 different individuals, which is consistent with the number of Procapra przewalskii subadults in Jiangxigou Procapra przewalskii Rescue Center.

The present disclosure further provides use of the primer combination of SSR molecular marker or the kit in detection for a population genetic diversity of Procapra przewalskii.

In the present disclosure, a combination of the above 26 SSR molecular marker primers of Procapra przewalskii can stably amplify a target product, and are highly polymorphic, and can be used for population genetic diversity detection, population genetic structure analysis, evolution, and kinship research of the Procapra przewalskii.

The present disclosure further provides a method for individual identification of Procapra przewalskii, including the following steps: genomic DNAs are extracted from samples of the Procapra przewalskii, subjected to PCR amplification with the primer pairs, then a capillary electrophoresis was performed to determine genotypes at loci of the SSR molecular markers for individual identification; and when the genotypes at loci of the SSR molecular markers are the same or only one genotype at one locus is different, the samples are determined to come from the same individual.

In the present disclosure, the sample includes preferably a feces or a tissue, more preferably the feces; and the tissue more preferably includes fur, muscle, and blood.

The technical solution provided by the present disclosure will be described in detail below with reference to the examples, but they should not be construed as limiting the claimed scope of the present disclosure.

In the examples of the present disclosure, experimental samples, experimental reagents, and experimental instrument and equipment are as follows:

### 1. Experimental Samples

15 feces samples of Procapra przewalskii used in polymorphic SSR locus screening are collected from Haiyan County, Gonghe County, and Gangcha County in Qinghai Province. Fresh faces samples collected in the field are immediately stored in liquid nitrogen, and transferred to a −80° C. refrigerator after returning to the laboratory for storage until a total genomic DNA extraction.

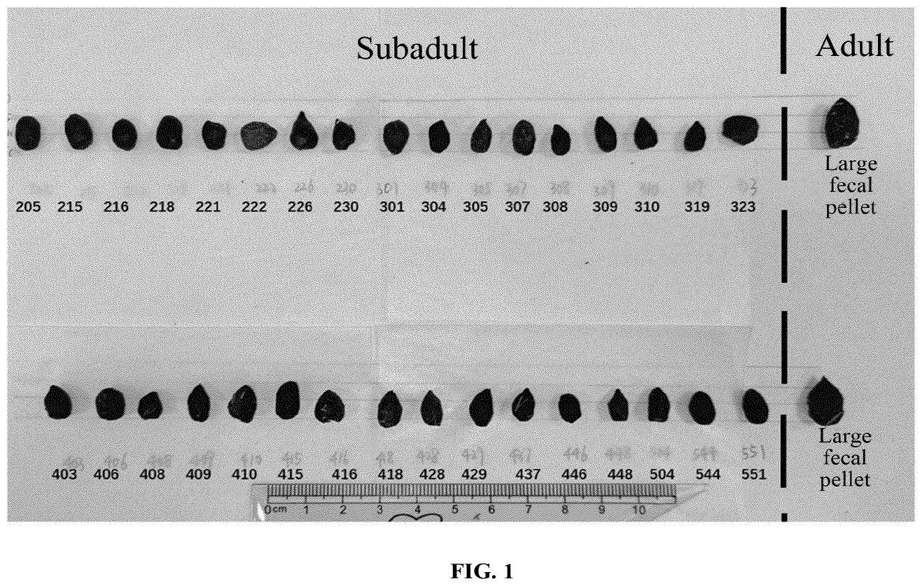

Samples for individual identification are collected from Jiangxigou Procapra przewalskii Rescue Center. In April 2022, the feces samples of Procapra przewalskii subadults are collected at the rescue center for 4 consecutive days and collected twice a day to ensure that feces samples of all Procapra przewalskii subadults are collected. A total of 33 feces samples of subadults are collected in 4 days (as shown in FIG. 1).

### 2. Experimental Reagents

QIAamp Fast DNA Stool Mini Kit, TaKaRa Ex Taq Hot Start Version, absolute ethanol, 100 bp DNA Ladder, 6×loading buffer, 50×TAE, agarose, and Ethidium bromide (EB).

### 3. Experimental Instruments and Equipment

ABI 3730XL genetic analyzer, ABI Veriti temperature-gradient PCR instrument, Nanodrop 2000C spectrophotometer, Bio-rad electrophoresis system, Bio-rad gel imaging system, Millipore pure water system, high-speed refrigerated centrifuge, autoclave, 4° C. refrigerator, −20° C. refrigerator, −80° C. refrigerator, vortex shaker, ice maker, constant-temperature water bath, electronic balance, eppendorf pipette, and microwave oven.

### Example 1

Extraction and Detection of Total Genomic DNA from Feces of Procapra przewalskii

A method by kit was adopted, and the kit used was QIAamp Fast DNA Stool (51604).

Preparation phase: it was ensured that Buffer AW1 and Buffer AW2 had been prepared according to the instructions on the kit label. The buffers were mixed well before use. If a precipitate formed in Buffer ASL or Buffer AL, the precipitate was dissolved by heating in a 70° C. water bath.
(1) DNA extraction: 180 mg to 220 mg of a feces sample was added into 2 mL centrifuge tubes and placed on ice for pretreatment.
(2) 1 mL of Inhibit EX Buffer was added to the sample, and vortexed intermittently for 1 min to 2 min until the sample was completely homogenized.
(3) The homogenized product was centrifuged at a full speed of 14,000 rpm for 1.5 min to make the feces settle to a bottom of the centrifuge tube.
(4) 25 μL proteinase K was added to a new 2 mL microcentrifuge tube.
(5) 600 μL of a supernatant from the centrifuge tube in step 3 was pipetted into a 2 mL centrifuge tube with proteinase K.
(6) 600 μL of Buffer AL was added and vortexed for 15 s to fully mix a resulting solution.
(7) The solution was incubated at 70° C. for 10 min, and mixed by inversion 1 or 2 times during incubation to reduce droplets on the cap of a centrifuge collection tube, then was placed and cooled on ice.
(8) 600 μL of pre-cooled absolute ethanol solution was added to the lysate, and vortexed to mix well and reduce droplets on the cap of a centrifuge collection tube.
(9) 600 μL of a solution obtained in the step (8) was added into an adsorption column (the adsorption column was put into a 2 mL collection tube). Centrifugation was conducted at full speed of 14,000 rpm for 1.5 min, a waste liquid generated was discarded, and the adsorption column was placed into a new collection tube.
(10) The cap of the adsorption column was opened and another 600 μL of lysate was added. Centrifugation was conducted at full speed of 14,000 rpm for 1.5 min, a waste liquid generated was discarded, and the adsorption column was placed into a new collection tube.
(11) The step (10) was repeated to load the third 600 μL lysate onto the adsorption column. Centrifugation was conducted at full speed of 14,000 rpm for 1.5 min, a waste liquid generated was discarded, and the adsorption column was put into a new collection tube.
(12) 500 μL Buffer AW1 was added to the adsorption column. The centrifuge tube was sealed to allow centrifugation at full speed of 14,000 rpm for 1.5 min. The centrifuge tube was taken out and put into a new 2 mL collection tube. The old collection tube and fluid therein were discarded.
(13) 500 μL Buffer AW2 was added to the adsorption column. The centrifuge tube was sealed to allow centrifugation at full speed of 14,000 rpm for 3 min. The centrifuge tube was taken out and put into a new 2 mL collection tube. The old collection tube and fluid therein were discarded.
(14) The centrifuge tube was taken out and put into a new 2 mL collection tube to allow centrifugation at full speed of 14,000 rpm for 3 min. The adsorption column was transferred to a new 1.5 mL centrifuge tube, added with 200 μL of Buffer ATE carefully, allowed to stand at room temperature for 2 min, and centrifuged at full speed of 14,000 rpm for 2 min to elute the DNA. The DNA samples in the 1.5 mL centrifuge tube were quickly placed in a −20° C. refrigerator.

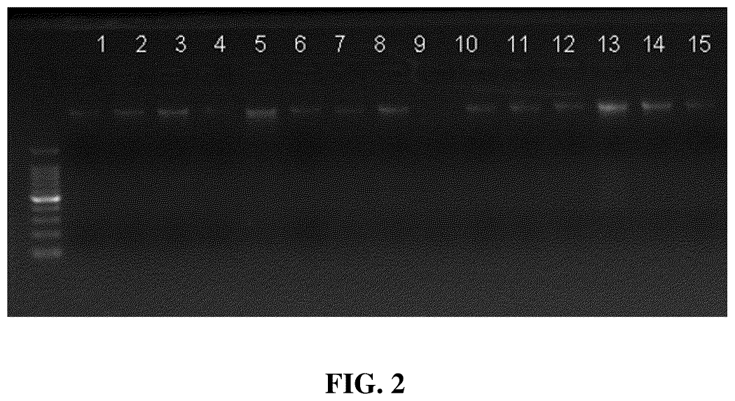

After the DNA extraction was completed, the DNA concentration was detected with a Nanodrop 2000C spectrophotometer, and the DNA quality was detected by 1% agarose gel electrophoresis. The specific results were shown in FIG. 2 and Table 1.

TABLE 1
Detection results of concentration of total genomic DNA

| | DNA | | DNA | | DNA |
|---|---|---|---|---|---|
| | concen- | | concen- | | concen- |
| Sample | tration | Sample | tration | Sample | tration |
| NO: | (ng/μL) | NO: | (ng/μL) | NO: | (ng/μL) |
| 2501 | 9.5 | 3302 | 57.7 | 018 | 7.3 |
| 2502 | 11.0 | 3304 | 5.5 | 022 | 15.1 |
| 3201 | 15.1 | 3306 | 51.3 | 023 | 26.35 |
| 3203 | 22.4 | 3309 | 2.6 | 024 | 19.35 |
| 3301 | 3.1 | 009 | 2.8 | 026 | 14.1 |

As shown in FIG. 2, the extracted total genomic DNA showed a brighter main band, indicating that the DNA quality was relatively high. As shown in Table 1, the total genomic DNA of the Procapra przewalskii had a concentration between 2.6 (sample 3309) to 57.7 (sample 3302) ng/μL, and could be used for subsequent PCR experiments.

### Example 2

### SSR Primer Screening and Polymorphism Detection

### 1. SSR Selection and Primer Design

The whole genome of Procapra przewalskii was scanned with MISA microsatellite screening software, and the SSR loci with a repeat sequence of 4 bp to 6 bp and a repeat number greater than 8 were identified, and a total of 1,632 SSR loci meeting the conditions were obtained. From the SSR loci with a repeat unit of 4 bp and a repeat number of 10 to 70, 5 to 6 loci were randomly selected for each chromosome, totaling 150 loci. With the upstream and downstream sequences of SSR loci, primers were designed by Primer3 software.

The primer design followed the following principles: a primer length should be 18 bp to 23 bp; a Tm value of the primer should be 55° C. to 63° C., and an optimum temperature was about 59° C.; a Tm difference between the forward and reverse primers was less than or equal to 5° C.

Among the 137 loci for which primers were successfully designed, 3 to 5 loci were randomly selected for each chromosome, and a total of 100 pairs of primers were selected. These primers were synthesized by Sangon Biotech (Shanghai) Co., Ltd. for subsequent PCR amplification.

### 2. PCR Amplification and Specificity Detection

A PCR reaction system (20 μL) was shown in Table 2. The PCR amplification was conducted on an ABI Veriti temperature-gradient PCR instrument, and the conditions for a first round of PCR amplification were shown in Table 3.

TABLE 2
PCR reaction system (20 μL)

| | | |
|---|---|---|
| Reagent | Concentration | Consumption (μL) |

| | | | |
|---|---|---|---|
| Template DNA | 50 | ng/μL | 2 |
| Primer F | 10 | mM | 1 |
| Primer R | 10 | mM | 1 |
| dNTP Mixture | 10 | μM | 2 |

| | | |
|---|---|---|
| Taq Buffer (MgCl₂ plus) | 10× | 2.5 |

| | | | |
|---|---|---|---|
| TaKaRa Ex Taq HS | 5 | U/μL | 0.2 |

| | | |
|---|---|---|
| ddH₂O | — | 11.3 |

TABLE 3
PCR reaction program

| Serial | | | | |
|---|---|---|---|---|
| number | Program | Temperature | Time | |
| 1 | Pre-denaturation | 95° C. | 5 | min |
| 2 | Denaturation | 94° C. | 30 | sec |
| 3 | Annealing | 60° C. | 30 | sec |
| 4 | Extension | 72° C. | 30 | sec |
| 5 | Cycling for | — | 10 | cycles |
| | step 2-4 | | | |
| 6 | Denaturation | 94° C. | 30 | sec |
| 7 | Annealing | 55° C. | 30 | sec |
| 8 | Extension | 72° C. | 30 | sec |
| 9 | Cycling for | — | 35 | cycles |
| | step 6-8 | | | |
| 10 | Repair and | 72° C. | 8 | min |
| | extension | | | |

3 μL of the PCR amplification product was subjected to 1.5% agarose gel electrophoresis at 220 V for 15 min to screen for SSR loci capable of specific amplification.

The reaction conditions corresponding to unsatisfactory amplification results were optimized. If the electrophoresis results showed lighter bands or tailing bands, the PCR amplification conditions were optimized. When the bands were shallower, the number of cycles increased from 35 to 37 cycles. In the case of tailing of the electrophoresis bands, the annealing temperature was increased from 60° C. to 63° C. If multiple bands appeared, it indicated that the microsatellite locus was not specific. When no band appears, the annealing temperature was decreased from 60° C. to 57° C. PCR reaction conditions were optimized for 8 loci (Table 4).

TABLE 4
Optimization of PCR reaction program

| Primer name | PCR program |
|---|---|
| PR-53 | 10 cycles at 57° C.; 35 cycles at 55° C. |
| PR-22 | 10 cycles at 63° C.; 35 cycles at 55° C. |
| PR-25 | 10 cycles at 63° C.; 35 cycles at 55° C. |
| PR-26 | 10 cycles at 63° C.; 35 cycles at 55° C. |
| PR-35 | 10 cycles at 63° C.; 35 cycles at 55° C. |
| PR-48 | 10 cycles at 63° C.; 35 cycles at 55° C. |
| PR-44 | 10 cycles at 60° C.; 37 cycles at 55° C. |
| PR-98 | 10 cycles at 60° C.; 37 cycles at 55° C. |

### 3. Polymorphism Detection by Capillary Electrophoresis

In 15 individuals, 3 DNA templates were selected for PCR amplification of 100 SSR loci. PCR products were subjected to agarose gel electrophoresis, and 60 specifically amplified SSR loci were selected.

The fluorescent adapter primer and forward primer were used for PCR amplification, and the amplification system and conditions were the same as those described above. The polymorphism of the amplified product was detected by capillary electrophoresis on the 3037XL Genetic Analyzer, the specific steps were as follows:
(1) HiDi and 500 internal standards were mixed at 130:1 to obtain a mix.
(2) The mix was dispensed in a 96-well reaction plate, and 10 μL of the mix was added to each well.
(3) 0.5 μL of a PCR product was added to the 96-well plate to start a centrifugation, and the centrifugation was stopped at 4,000 rpm.
(4) A mixing plate was heated at 95° C. for 5 min with a metal bath heater to pre-denature the mixing plate, and placed at −20° C. immediately after taking out.
(5) After cooling, the mixing plate was taken centrifuged at 4,000 rpm, thawed, and mixed.
(6) The capillary electrophoresis was conducted with a 3037XL genetic analyzer.
(7) The off-machine results were acquired and analyzed.

26 pairs of primers with high polymorphism were detected by the capillary electrophoresis, and the primer characteristics were shown in Table 5.

TABLE 5
Primer characteristics of 26 pairs of SSR loci with high polymorphism

| | | | | Sequence |
|---|---|---|---|---|
| | | | | information |
| | | | | (serial |
| | | | Amplified | number in |
| Primer | | | product | sequence |
| number | Primer sequence 5′→3′ | Repeat unit | size | listing) |
| PR-6 | F: AGCTAGACCCTAAATCCTTGTGT | (ATAG) 11 | 222 | 1 |
| | R: ACTCCTCTCACGTCTTCAGC | | | 2 |
| PR-7 | F: TCCTACACATGTGCTTGGAA | (ATCT) 13 | 232 | 3 |
| | R: ATCCATGCCCTAATGCCTCC | | | 4 |
| PR-8 | F: ACTTGGGGAAATCTCAAAAGTCT | (AAAG) 10 | 172 | 5 |
| | R: GGCAAAAGCCTTAGAGAAATGC | | | 6 |
| PR-10 | F: ATCTATGGGGTCGCACAGAG | (ATAG) 10 | 214 | 7 |
| | R: ACCAGAGTTCAGAACCAGTGT | | | 8 |
| PR-12 | F: TCCCCATTACCACACCTAAGT | (AAAC) 10 | 141 | 9 |
| | R: ACATCCAGGAGAGTGTCAGC | | | 10 |
| PR-14 | F: AGATGAACTGCTCTGGGAAG | (TATG) 13 | 186 | 11 |
| | R: AATGGGAAAGTATGAAAGGCA | | | 12 |
| PR-16 | F: GTTTCTGCCCTCAATCTGTT | (TAGA) 11 | 232 | 13 |
| | R: AGACTTTTACATCTCAGCTAAGC | | | 14 |
| PR-22 | F: TCAGATCTCCCTCAATGCAGG | (ATAG) 10 | 143 | 15 |
| | R: GTGTTAGTTTCAGGTGTAGAACA | | | 16 |
| PR-25 | F: CCAAGAGTGAAGTTGTGCCA | (AGGA) 11 | 178 | 17 |
| | R: AACCAAGTGCTCTGTTTCAG | | | 18 |
| PR-26 | F: AAACTGGAATGGGTTGCCAC | (AGAT) 12 | 210 | 19 |
| | R: AGGGATGATGTATTAGTTAGGGT | | | 20 |
| PR-28 | F: ACCCAATTTCGAGCTTCCAG | (ATAG) 11 | 132 | 21 |
| | R: TGTACTAGTTTTCCAGAGCTGC | | | 22 |
| PR-30 | F: ATTCTCTCGGGCCATCTGTG | (ATAG) 12 | 200 | 23 |
| | R: CACTGCTGGAATGGTCAAGG | | | 24 |
| PR-40 | F: CCGTTCTCCAGGGGAATCTT | (AGAT) 11 | 182 | 25 |
| | R: TTCACAGGGCCTTCAGAAAC | | | 26 |
| PR-42 | F: TAGAGCCCAATCCAAGGTCC | (ATAG) 11 | 219 | 27 |
| | R: TGCAGAGCCATCAGTTAAGAC | | | 28 |
| PR-46 | F: CTCTGTCCTCCGCTGTCTC | (TAGA) 12 | 211 | 29 |
| | R: CAAATTGCCTTGTCCCTGCC | | | 30 |
| PR-53 | F: TCCCTGATCTGTAACAAATGTG | (AGGA) 11 | 168 | 31 |
| | R: CCTTAGCGTCACACACACAG | | | 32 |
| PR-58 | F: CAGAGTCGGTCATGATGGGA | (AGGA) 12 | 203 | 33 |
| | R: TGCTTCCGACCAAGTGTTTG | | | 34 |
| PR-63 | F: GGGGCATCACAATTCCATTCT | (AGAT) 11 | 230 | 35 |
| | R: TCCAACTTTCAGACCATAAATCC | | | 36 |
| PR-64 | F: CCCTCTCTGTTGTAGTTGTTGG | (TCTT) 10 | 150 | 37 |
| | R: ACAATCCTGCAAAAGCCCTC | | | 38 |
| PR-65 | F: GGCAAGTGAGGTGATGAAGC | (AAAG) 10 | 179 | 39 |
| | R: TGGCCTTCTAGAGTTGTTTTGC | | | 40 |
| PR-69 | F: AGCAATGAAGACCAGCATAACC | (AAAG) 10 | 232 | 41 |
| | R: CCCATCAATCTTTGCAGTCCC | | | 42 |
| PR-71 | F: TTTGCTGCCTCGACAAACAG | (TCCA) 11 | 208 | 43 |
| | R: TCCCGCATTACAAGCAGATTC | | | 44 |
| PR-72 | F: TGGTTCTGTACAAGCTTCAG | (AAAG) 10 | 157 | 45 |
| | R: CAGTCTGGATTGAGGGGTCT | | | 46 |
| PR-85 | F: GTTCCAAGCTAAGCATTTAAGGC | (ATAG) 12 | 231 | 47 |
| | R: GGGAACACATGTACACCCGT | | | 48 |
| PR-86 | F: CGCTTTACCATCTCAGCCAC | (ATAG) 13 | 135 | 49 |
| | R: AGTCATCCATCGGTCCATGT | | | 50 |
| PR-97 | F: TGATCACAGAACCCCACAAGT | (ACAT) 10 | 202 | 51 |
| | R: TAGGGGAAGCCTGTTAAGCA | | | 52 |

The 26 polymorphic SSR loci were further analyzed for polymorphism.

### 4. Polymorphism Analysis of SSR Loci

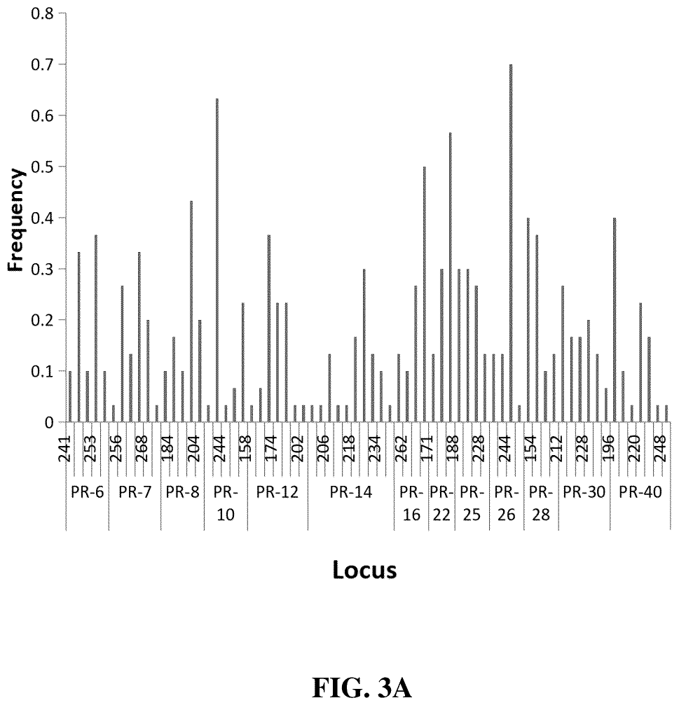

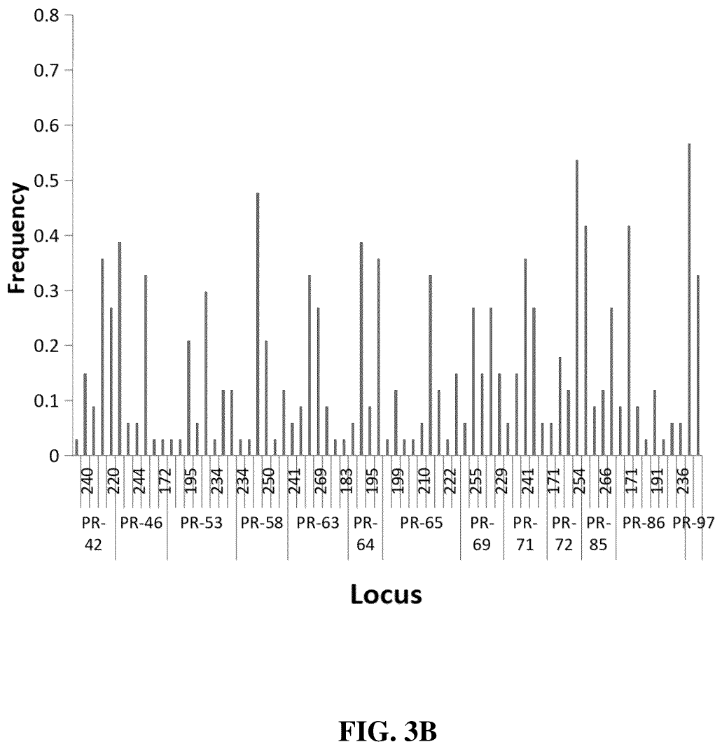

The allele number Nₐ, effective allele number Nₑ, observed heterozygosity Hₒ, and expected heterozygosity Hₑ of 15 samples in 26 pairs of primers were calculated using POPGENE1.31. A polymorphism information content PIC was calculated with PowerMarker3.25. The polymorphisms of 26 SSR loci were evaluated with analysis software PowerMarker3.25. The detailed results were shown in Table 6 and FIGS. 3A-B.

TABLE 6
Polymorphic characteristics of 26 pairs
of primers successfully amplified

| | | Effective | Observed | Expected | |
|---|---|---|---|---|---|
| | Allele | allele | hetero- | hetero- | PIC of |
| Primer | number | number | zygosity | zygosity | polymor- |
| number | Nₐ | Nₑ | Hₒ | Hₑ | phism |
| PR-6 | 5 | 3.6290 | 0.7333 | 0.7494 | 0.6792 |
| PR-7 | 6 | 4.1284 | 0.8667 | 0.7839 | 0.7184 |
| PR-8 | 5 | 3.6290 | 0.2667 | 0.7494 | 0.6863 |
| PR-10 | 5 | 2.1635 | 0.4667 | 0.5563 | 0.4880 |
| PR-12 | 7 | 3.9823 | 0.7333 | 0.7747 | 0.7099 |
| PR-14 | 10 | 5.9211 | 0.8667 | 0.8598 | 0.8122 |
| PR-16 | 4 | 2.8662 | 0.7333 | 0.6736 | 0.5974 |
| PR-22 | 3 | 2.3316 | 0.4000 | 0.5908 | 0.4987 |
| PR-25 | 4 | 3.7190 | 0.7333 | 0.7563 | 0.6804 |
| PR-26 | 4 | 1.8987 | 0.4667 | 0.4897 | 0.4367 |
| PR-28 | 4 | 3.1034 | 0.6000 | 0.7011 | 0.6180 |
| PR-30 | 6 | 5.2941 | 1.0000 | 0.8391 | 0.7840 |
| PR-40 | 7 | 3.9130 | 1.0000 | 0.7701 | 0.7086 |
| PR-42 | 5 | 3.4615 | 0.8667 | 0.7356 | 0.6622 |
| PR-46 | 6 | 3.0000 | 0.6667 | 0.6897 | 0.6089 |
| PR-53 | 8 | 4.7872 | 0.6000 | 0.8184 | 0.7634 |
| PR-58 | 6 | 2.7778 | 0.6667 | 0.6621 | 0.5946 |
| PR-63 | 7 | 3.9823 | 0.6667 | 0.7747 | 0.7122 |
| PR-64 | 4 | 2.7607 | 0.4000 | 0.6598 | 0.5676 |
| PR-65 | 9 | 4.8387 | 0.4667 | 0.8207 | 0.7701 |
| PR-69 | 5 | 4.1667 | 0.8000 | 0.7862 | 0.7202 |
| PR-71 | 5 | 3.4884 | 0.6667 | 0.7379 | 0.6657 |
| PR-72 | 4 | 2.3684 | 0.7333 | 0.5977 | 0.5310 |
| PR-85 | 4 | 2.9801 | 0.4667 | 0.6874 | 0.6078 |
| PR-86 | 8 | 3.7500 | 0.5333 | 0.7586 | 0.7102 |
| PR-97 | 2 | 1.8672 | 0.3333 | 0.4805 | 0.3566 |

The greater the number of alleles at each locus, the more polymorphic the population could be. In the present disclosure, a total of 143 alleles were detected in amplified products of the 26 pairs of primers, and the number of alleles ranged from 2 (PR-97) to 10 (PR-14).

The Polymorphism information content (PIC) was calculated through the allele frequency, and could reflect the diversity degree of the SSR locus. When PIC>0.5, it indicated that the locus had high diversity and belonged to highly polymorphic locus; when PIC<0.25, it indicated that the locus had poor diversity level, and belonged to low polymorphic locus; when the PIC was between 0.25 and 0.5, it indicated that the locus had medium diversity level and belonged to the moderately polymorphic locus. In the present disclosure, 22 of the 26 pairs of SSR primers showed highly polymorphic loci, and an overall PIC was 0.3566 (PR-97) to 0.8122 (PR-14), with an average of 0.7431, indicating that the SSR locus of the present disclosure had a high polymorphism.

The higher the expected heterozygosity (Hₑ) of the SSR locus was, the lower the genetic consistency of the population could be, that is, the higher the population genetic diversity was. In the present disclosure, the Hₑ of the 26 SSR loci ranged from 0.4805 (PR-97) to 0.8598 (PR-14), with an average of 0.7117±0.1008; while the Hₒ ranged from 0.2667 (PR-8) to 1.0000 (PR-30, PR-40), with an average of 0.6436±0.1968. In summary, the allele number after amplification by the 26 pairs of primers of the present disclosure was 5.5±1.8815, and the effective allele number was 3.4926±1.0190. The results indicated that the Procapra przewalskii population had a high genetic diversity.

As shown in FIGS. 3A-B, the highest allele frequency of the 26 SSR loci was 0.7, and the lowest allele frequency was 0.03333.

It was seen that in the present disclosure, the primers could stably amplify a target product, and were highly polymorphic, and could be used for population genetic diversity detection, population genetic structure analysis, evolution, and kinship research of the Procapra przewalskii.

### Example 3

### Analysis of Individual Identification Ability of SSR Loci

### 1. Determination of the Probability of Identity of the SSR Loci

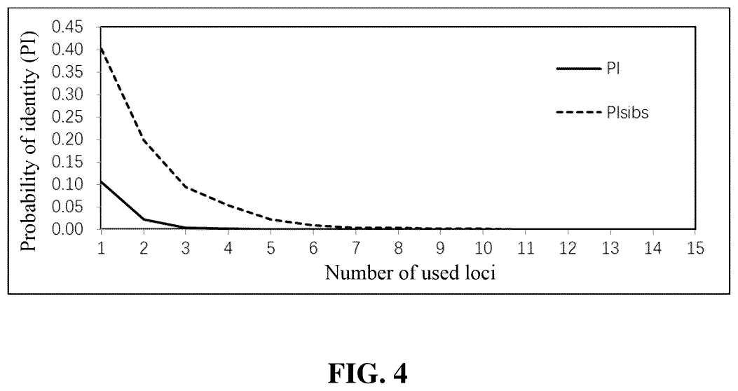

Probability of identity value refers to the probability that two randomly selected individuals in a population have the same genotype. This method is used to determine whether the number of SSR loci used can achieve individual identification. The appearance of the probability of identity (PI) value and probability of identity between siblings (PIsibs) value can obtain a conservative lower limit of the number of loci required to complete individual identification. In the present disclosure, according to the detection results of SSR polymorphic loci in 15 samples, GenAlEx V6.502 was used to analyze the genotypes of 26 loci in 15 individuals, and the probability of identity values (PI and PIsibs) were calculated. The specific results were shown in FIG. 4.

As shown in FIG. 4, the PI values of 15 SSR loci, PR-6, PR-8, PR-12, PR-22, PR-25, PR-30, PR-40, PR-58, PR-63, PR-69, PR-71, PR-72, PR-85, PR-86, and PR-97, were 8.9020×10⁻¹¹, indicating that when using this combination for individual identification, if 2 Procapra przewalskii individuals were randomly selected, the probability of the same haplotype was 8.9020×10⁻¹¹. The PIsibs value was 4.1834×10⁻⁵, indicating that the probability of having the same haplotype in any two Procapra przewalskii individuals for this combination of SSR loci was 4.1834×10⁻⁵ considering the kinship. This combination could achieve an identification rate of 1/10,000 for individual identification. The current population of Procapra przewalskii was only a few thousand, and the identification rate of 1/10,000 could meet the needs of individual identification for Procapra przewalskii. It was seen that the combination of SSR loci could meet the individual identification needs of Procapra przewalskii.

2. Individual Identification of Procapra przewalskii Subadults in Jiangxigou Procapra przewalskii Rescue Center

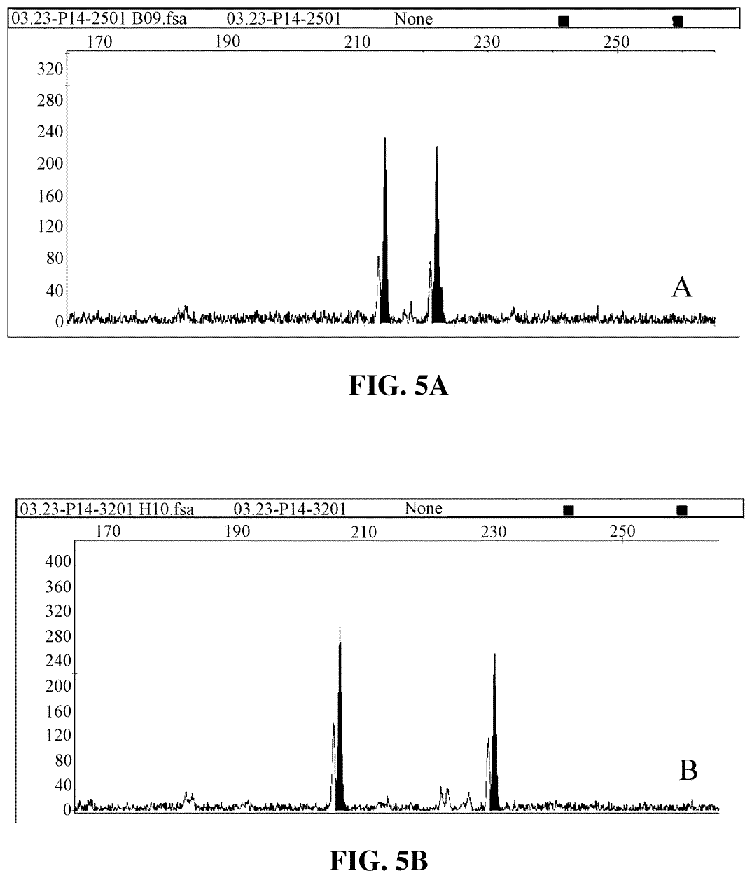

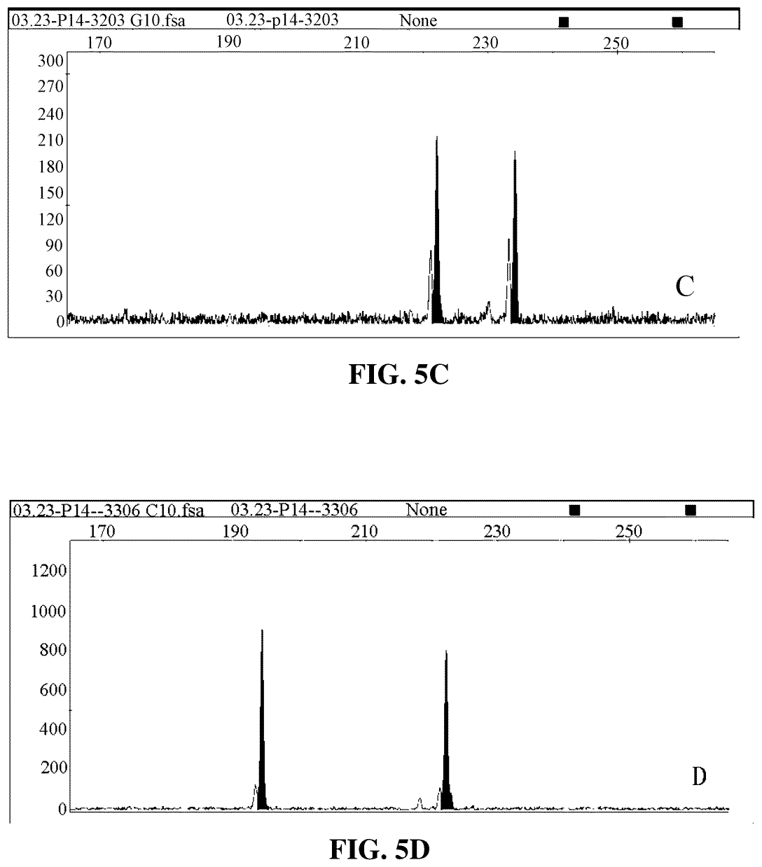

With the obtained 15 SSR loci, 33 Procapra przewalskii subadults collected from Jiangxigou Procapra przewalskii Rescue Center were subjected to individual identification by PCR amplification and capillary electrophoresis. Some electrophoresis results were shown in FIGS. 5A-D. The Microsatellite Tool kit program was used to search for matching genotypes in the data, and the matching genotypes were compared with the actual number of subadult animals in the rescue center. In this way, the identification ability and accuracy of the developed SSR loci were detected and identified, and the specific results were shown in Table 7.

The following principle was obeyed when identifying individuals: if the genotypes at all microsatellite loci in individuals were the same or only one genotype at one locus was different, then the individuals were regarded as the same individual.

TABLE 7
Comparison results of genotypes of 15 SSR loci in 33
feces samples of Procapra przewalskii subadults

| Sample 1 | Sample 2 | Matching probability |
|---|---|---|
| 205 | 215 | 53.33% |
| 205 | 216 | 46.67% |
| 205 | 218 | 53.33% |
| 205 | 221 | 53.33% |
| 205 | 222 | 53.33% |
| 205 | 226 | 40.00% |
| 205 | 230 | 46.67% |
| 205 | 301 | 63.33% |
| 205 | 304 | 43.33% |
| 205 | 305 | 60.00% |
| 205 | 307 | 53.33% |
| 205 | 308 | 63.33% |
| 205 | 309 | 56.67% |
| 205 | 310 | 50.00% |
| 205 | 319 | 63.33% |
| 205 | 323 | 46.67% |
| 205 | 403 | 53.33% |
| 205 | 406 | 46.67% |
| 205 | 408 | 60.00% |
| 205 | 409 | 50.00% |
| 205 | 410 | 46.67% |
| 205 | 415 | 50.00% |
| 205 | 416 | 63.33% |
| 205 | 418 | 53.33% |
| 205 | 428 | 60.00% |
| 205 | 429 | 63.33% |
| 205 | 437 | 43.33% |
| 205 | 446 | 43.33% |
| 205 | 448 | 60.00% |
| 205 | 504 | 60.00% |
| 205 | 544 | 63.33% |
| 205 | 551 | 60.00% |
| 215 | 216 | 50.00% |
| 215 | 218 | 33.33% |
| 215 | 221 | 56.67% |
| 215 | 222 | 63.33% |
| 215 | 226 | 60.00% |
| 215 | 230 | 40.00% |
| 215 | 301 | 43.33% |
| 215 | 304 | 53.33% |
| 215 | 305 | 63.33% |
| 215 | 307 | 56.67% |
| 215 | 308 | 53.33% |
| 215 | 309 | 63.33% |
| 215 | 310 | 63.33% |
| 215 | 319 | 53.33% |
| 215 | 323 | 40.00% |
| 215 | 403 | 33.33% |
| 215 | 406 | 40.00% |
| 215 | 408 | 63.33% |
| 215 | 409 | 96.67% |
| 215 | 410 | 50.00% |
| 215 | 415 | 60.00% |
| 215 | 416 | 53.33% |
| 215 | 418 | 46.67% |
| 215 | 428 | 70.00% |
| 215 | 429 | 43.33% |
| 215 | 437 | 40.00% |
| 215 | 446 | 53.33% |
| 215 | 448 | 63.33% |
| 215 | 504 | 60.00% |
| 215 | 544 | 56.67% |
| 215 | 551 | 53.33% |
| 216 | 218 | 40.00% |
| 216 | 221 | 66.67% |
| 216 | 222 | 63.33% |
| 216 | 226 | 66.67% |
| 216 | 230 | 26.67% |
| 216 | 301 | 60.00% |
| 216 | 304 | 43.33% |
| 216 | 305 | 73.33% |
| 216 | 307 | 50.00% |
| 216 | 308 | 53.33% |
| 216 | 309 | 50.00% |
| 216 | 310 | 40.00% |
| 216 | 319 | 53.33% |
| 216 | 323 | 26.67% |
| 216 | 403 | 40.00% |
| 216 | 406 | 30.00% |
| 216 | 408 | 63.33% |
| 216 | 409 | 50.00% |
| 216 | 410 | 96.67% |
| 216 | 415 | 53.33% |
| 216 | 416 | 46.67% |
| 216 | 418 | 43.33% |
| 216 | 428 | 63.33% |
| 216 | 429 | 60.00% |
| 216 | 437 | 70.00% |
| 216 | 446 | 43.33% |
| 216 | 448 | 63.33% |
| 216 | 504 | 60.00% |
| 216 | 544 | 53.33% |
| 216 | 551 | 53.33% |
| 218 | 221 | 36.67% |
| 218 | 222 | 40.00% |
| 218 | 226 | 26.67% |
| 218 | 230 | 40.00% |
| 218 | 301 | 43.33% |
| 218 | 304 | 46.67% |
| 218 | 305 | 43.33% |
| 218 | 307 | 60.00% |
| 218 | 308 | 33.33% |
| 218 | 309 | 40.00% |
| 218 | 310 | 50.00% |
| 218 | 319 | 33.33% |
| 218 | 323 | 40.00% |
| 218 | 403 | 100.00% |
| 218 | 406 | 43.33% |
| 218 | 408 | 56.67% |
| 218 | 409 | 30.00% |
| 218 | 410 | 36.67% |
| 218 | 415 | 33.33% |
| 218 | 416 | 40.00% |
| 218 | 418 | 46.67% |
| 218 | 428 | 43.33% |
| 218 | 429 | 43.33% |
| 218 | 437 | 46.67% |
| 218 | 446 | 46.67% |
| 218 | 448 | 56.67% |
| 218 | 504 | 46.67% |
| 218 | 544 | 50.00% |
| 218 | 551 | 56.67% |
| 221 | 222 | 46.67% |
| 221 | 226 | 70.00% |
| 221 | 230 | 43.33% |
| 221 | 301 | 70.00% |
| 221 | 304 | 50.00% |
| 221 | 305 | 56.67% |
| 221 | 307 | 53.33% |
| 221 | 308 | 63.33% |
| 221 | 309 | 46.67% |
| 221 | 310 | 40.00% |
| 221 | 319 | 63.33% |
| 221 | 323 | 43.33% |
| 221 | 403 | 36.67% |
| 221 | 406 | 43.33% |
| 221 | 408 | 56.67% |
| 221 | 409 | 56.67% |
| 221 | 410 | 66.67% |
| 221 | 415 | 46.67% |
| 221 | 416 | 50.00% |
| 221 | 418 | 56.67% |
| 221 | 428 | 66.67% |
| 221 | 429 | 70.00% |
| 221 | 437 | 53.33% |
| 221 | 446 | 50.00% |
| 221 | 448 | 56.67% |
| 221 | 504 | 56.67% |
| 221 | 544 | 73.33% |
| 221 | 551 | 53.33% |
| 222 | 226 | 63.33% |
| 222 | 230 | 30.00% |
| 222 | 301 | 43.33% |
| 222 | 304 | 56.67% |
| 222 | 305 | 63.33% |
| 222 | 307 | 46.67% |
| 222 | 308 | 46.67% |
| 222 | 309 | 56.67% |
| 222 | 310 | 46.67% |
| 222 | 319 | 46.67% |
| 222 | 323 | 30.00% |
| 222 | 403 | 40.00% |
| 222 | 406 | 33.33% |
| 222 | 408 | 66.67% |
| 222 | 409 | 63.33% |
| 222 | 410 | 63.33% |
| 222 | 415 | 56.67% |
| 222 | 416 | 66.67% |
| 222 | 418 | 56.67% |
| 222 | 428 | 56.67% |
| 222 | 429 | 43.33% |
| 222 | 437 | 53.33% |
| 222 | 446 | 56.67% |
| 222 | 448 | 66.67% |
| 222 | 504 | 66.67% |
| 222 | 544 | 50.00% |
| 222 | 551 | 50.00% |
| 226 | 230 | 40.00% |
| 226 | 301 | 63.33% |
| 226 | 304 | 53.33% |
| 226 | 305 | 60.00% |
| 226 | 307 | 53.33% |
| 226 | 308 | 66.67% |
| 226 | 309 | 56.67% |
| 226 | 310 | 46.67% |
| 226 | 319 | 66.67% |
| 226 | 323 | 40.00% |
| 226 | 403 | 26.67% |
| 226 | 406 | 43.33% |
| 226 | 408 | 53.33% |
| 226 | 409 | 60.00% |
| 226 | 410 | 66.67% |
| 226 | 415 | 60.00% |
| 226 | 416 | 60.00% |
| 226 | 418 | 50.00% |
| 226 | 428 | 56.67% |
| 226 | 429 | 63.33% |
| 226 | 437 | 53.33% |
| 226 | 446 | 53.33% |
| 226 | 448 | 53.33% |
| 226 | 504 | 60.00% |
| 226 | 544 | 63.33% |
| 226 | 551 | 50.00% |
| 230 | 301 | 43.33% |
| 230 | 304 | 36.67% |
| 230 | 305 | 30.00% |
| 230 | 307 | 46.67% |
| 230 | 308 | 40.00% |
| 230 | 309 | 40.00% |
| 230 | 310 | 36.67% |
| 230 | 319 | 40.00% |
| 230 | 323 | 100.00% |
| 230 | 403 | 40.00% |
| 230 | 406 | 96.67% |
| 230 | 408 | 40.00% |
| 230 | 409 | 40.00% |
| 230 | 410 | 26.67% |
| 230 | 415 | 30.00% |
| 230 | 416 | 50.00% |
| 230 | 418 | 46.67% |
| 230 | 428 | 36.67% |
| 230 | 429 | 43.33% |
| 230 | 437 | 36.67% |
| 230 | 446 | 36.67% |
| 230 | 448 | 40.00% |
| 230 | 504 | 43.33% |
| 230 | 544 | 50.00% |
| 230 | 551 | 40.00% |
| 301 | 304 | 40.00% |
| 301 | 305 | 56.67% |
| 301 | 307 | 60.00% |
| 301 | 308 | 80.00% |
| 301 | 309 | 46.67% |
| 301 | 310 | 46.67% |
| 301 | 319 | 80.00% |
| 301 | 323 | 43.33% |
| 301 | 403 | 43.33% |
| 301 | 406 | 46.67% |
| 301 | 408 | 53.33% |
| 301 | 409 | 40.00% |
| 301 | 410 | 56.67% |
| 301 | 415 | 43.33% |
| 301 | 416 | 50.00% |
| 301 | 418 | 50.00% |
| 301 | 428 | 60.00% |
| 301 | 429 | 100.00% |
| 301 | 437 | 43.33% |
| 301 | 446 | 40.00% |
| 301 | 448 | 53.33% |
| 301 | 504 | 53.33% |
| 301 | 544 | 70.00% |
| 301 | 551 | 56.67% |
| 304 | 305 | 50.00% |
| 304 | 307 | 56.67% |
| 304 | 308 | 43.33% |
| 304 | 309 | 46.67% |
| 304 | 310 | 50.00% |
| 304 | 319 | 43.33% |
| 304 | 323 | 36.67% |
| 304 | 403 | 46.67% |
| 304 | 406 | 40.00% |
| 304 | 408 | 63.33% |
| 304 | 409 | 50.00% |
| 304 | 410 | 43.33% |
| 304 | 415 | 53.33% |
| 304 | 416 | 53.33% |
| 304 | 418 | 60.00% |
| 304 | 428 | 46.67% |
| 304 | 429 | 40.00% |
| 304 | 437 | 56.67% |
| 304 | 446 | 100.00% |
| 304 | 448 | 63.33% |
| 304 | 504 | 56.67% |
| 304 | 544 | 56.67% |
| 304 | 551 | 46.67% |
| 305 | 307 | 56.67% |
| 305 | 308 | 56.67% |
| 305 | 309 | 66.67% |
| 305 | 310 | 50.00% |
| 305 | 319 | 56.67% |
| 305 | 323 | 30.00% |
| 305 | 403 | 43.33% |
| 305 | 406 | 33.33% |
| 305 | 408 | 63.33% |
| 305 | 409 | 63.33% |
| 305 | 410 | 70.00% |
| 305 | 415 | 66.67% |
| 305 | 416 | 56.67% |
| 305 | 418 | 43.33% |
| 305 | 428 | 63.33% |
| 305 | 429 | 56.67% |
| 305 | 437 | 60.00% |
| 305 | 446 | 50.00% |
| 305 | 448 | 63.33% |
| 305 | 504 | 80.00% |
| 305 | 544 | 60.00% |
| 305 | 551 | 53.33% |
| 307 | 308 | 56.67% |
| 307 | 309 | 56.67% |
| 307 | 310 | 73.33% |
| 307 | 319 | 56.67% |
| 307 | 323 | 46.67% |
| 307 | 403 | 60.00% |
| 307 | 406 | 50.00% |
| 307 | 408 | 66.67% |
| 307 | 409 | 53.33% |
| 307 | 410 | 46.67% |
| 307 | 415 | 43.33% |
| 307 | 416 | 56.67% |
| 307 | 418 | 56.67% |
| 307 | 428 | 53.33% |
| 307 | 429 | 60.00% |
| 307 | 437 | 50.00% |
| 307 | 446 | 56.67% |
| 307 | 448 | 66.67% |
| 307 | 504 | 50.00% |
| 307 | 544 | 63.33% |
| 307 | 551 | 63.33% |
| 308 | 309 | 50.00% |
| 308 | 310 | 53.33% |
| 308 | 319 | 100.00% |
| 308 | 323 | 40.00% |
| 308 | 403 | 33.33% |
| 308 | 406 | 43.33% |
| 308 | 408 | 50.00% |
| 308 | 409 | 50.00% |
| 308 | 410 | 50.00% |
| 308 | 415 | 50.00% |
| 308 | 416 | 50.00% |
| 308 | 418 | 46.67% |
| 308 | 428 | 60.00% |
| 308 | 429 | 80.00% |
| 308 | 437 | 43.33% |
| 308 | 446 | 43.33% |
| 308 | 448 | 50.00% |
| 308 | 504 | 53.33% |
| 308 | 544 | 66.67% |
| 308 | 551 | 46.67% |
| 309 | 310 | 53.33% |
| 309 | 319 | 50.00% |
| 309 | 323 | 40.00% |
| 309 | 403 | 40.00% |
| 309 | 406 | 40.00% |
| 309 | 408 | 60.00% |
| 309 | 409 | 63.33% |
| 309 | 410 | 53.33% |
| 309 | 415 | 66.67% |
| 309 | 416 | 73.33% |
| 309 | 418 | 40.00% |
| 309 | 428 | 60.00% |
| 309 | 429 | 46.67% |
| 309 | 437 | 40.00% |
| 309 | 446 | 46.67% |
| 309 | 448 | 60.00% |
| 309 | 504 | 56.67% |
| 309 | 544 | 53.33% |
| 309 | 551 | 60.00% |
| 310 | 319 | 53.33% |
| 310 | 323 | 36.67% |
| 310 | 403 | 50.00% |
| 310 | 406 | 36.67% |
| 310 | 408 | 63.33% |
| 310 | 409 | 60.00% |
| 310 | 410 | 40.00% |
| 310 | 415 | 40.00% |
| 310 | 416 | 53.33% |
| 310 | 418 | 43.33% |
| 310 | 428 | 60.00% |
| 310 | 429 | 46.67% |
| 310 | 437 | 46.67% |
| 310 | 446 | 50.00% |
| 310 | 448 | 63.33% |
| 310 | 504 | 50.00% |
| 310 | 544 | 43.33% |
| 310 | 551 | 70.00% |
| 319 | 323 | 40.00% |
| 319 | 403 | 33.33% |
| 319 | 406 | 43.33% |
| 319 | 408 | 50.00% |
| 319 | 409 | 50.00% |
| 319 | 410 | 50.00% |
| 319 | 415 | 50.00% |
| 319 | 416 | 50.00% |
| 319 | 418 | 46.67% |
| 319 | 428 | 60.00% |
| 319 | 429 | 80.00% |
| 319 | 437 | 43.33% |
| 319 | 446 | 43.33% |
| 319 | 448 | 50.00% |
| 319 | 504 | 53.33% |
| 319 | 544 | 66.67% |
| 319 | 551 | 46.67% |
| 323 | 403 | 40.00% |
| 323 | 406 | 96.67% |
| 323 | 408 | 40.00% |
| 323 | 409 | 40.00% |
| 323 | 410 | 26.67% |
| 323 | 415 | 30.00% |
| 323 | 416 | 50.00% |
| 323 | 418 | 46.67% |
| 323 | 428 | 36.67% |
| 323 | 429 | 43.33% |
| 323 | 437 | 36.67% |
| 323 | 446 | 36.67% |
| 323 | 448 | 40.00% |
| 323 | 504 | 43.33% |
| 323 | 544 | 50.00% |
| 323 | 551 | 40.00% |
| 403 | 406 | 43.33% |
| 403 | 408 | 56.67% |
| 403 | 409 | 30.00% |
| 403 | 410 | 36.67% |
| 403 | 415 | 33.33% |
| 403 | 416 | 40.00% |
| 403 | 418 | 46.67% |
| 403 | 428 | 43.33% |
| 403 | 429 | 43.33% |
| 403 | 437 | 46.67% |
| 403 | 446 | 46.67% |
| 403 | 448 | 56.67% |
| 403 | 504 | 46.67% |
| 403 | 544 | 50.00% |
| 403 | 551 | 56.67% |
| 406 | 408 | 36.67% |
| 406 | 409 | 40.00% |
| 406 | 410 | 26.67% |
| 406 | 415 | 33.33% |
| 406 | 416 | 53.33% |
| 406 | 418 | 50.00% |
| 406 | 428 | 36.67% |
| 406 | 429 | 46.67% |
| 406 | 437 | 40.00% |
| 406 | 446 | 40.00% |
| 406 | 448 | 36.67% |
| 406 | 504 | 46.67% |
| 406 | 544 | 53.33% |
| 406 | 551 | 40.00% |
| 408 | 409 | 60.00% |
| 408 | 410 | 63.33% |
| 408 | 415 | 46.67% |
| 408 | 416 | 56.67% |
| 408 | 418 | 53.33% |
| 408 | 428 | 63.33% |
| 408 | 429 | 53.33% |
| 408 | 437 | 60.00% |
| 408 | 446 | 63.33% |
| 408 | 448 | 100.00% |
| 408 | 504 | 56.67% |
| 408 | 544 | 56.67% |
| 408 | 551 | 70.00% |
| 409 | 410 | 50.00% |
| 409 | 415 | 60.00% |
| 409 | 416 | 53.33% |
| 409 | 418 | 43.33% |
| 409 | 428 | 70.00% |
| 409 | 429 | 40.00% |
| 409 | 437 | 40.00% |
| 409 | 446 | 50.00% |
| 409 | 448 | 60.00% |
| 409 | 504 | 60.00% |
| 409 | 544 | 53.33% |
| 409 | 551 | 53.33% |
| 410 | 415 | 50.00% |
| 410 | 416 | 46.67% |
| 410 | 418 | 43.33% |
| 410 | 428 | 63.33% |
| 410 | 429 | 56.67% |
| 410 | 437 | 66.67% |
| 410 | 446 | 43.33% |
| 410 | 448 | 63.33% |
| 410 | 504 | 56.67% |
| 410 | 544 | 50.00% |
| 410 | 551 | 53.33% |
| 415 | 416 | 53.33% |
| 415 | 418 | 36.67% |
| 415 | 428 | 56.67% |
| 415 | 429 | 43.33% |
| 415 | 437 | 36.67% |
| 415 | 446 | 53.33% |
| 415 | 448 | 46.67% |
| 415 | 504 | 63.33% |
| 415 | 544 | 50.00% |
| 415 | 551 | 36.67% |
| 416 | 418 | 53.33% |
| 416 | 428 | 60.00% |
| 416 | 429 | 50.00% |
| 416 | 437 | 43.33% |
| 416 | 446 | 53.33% |
| 416 | 448 | 56.67% |
| 416 | 504 | 60.00% |
| 416 | 544 | 56.67% |
| 416 | 551 | 60.00% |
| 418 | 428 | 53.33% |
| 418 | 429 | 50.00% |
| 418 | 437 | 53.33% |
| 418 | 446 | 60.00% |
| 418 | 448 | 53.33% |
| 418 | 504 | 53.33% |
| 418 | 544 | 66.67% |
| 418 | 551 | 46.67% |
| 428 | 429 | 60.00% |
| 428 | 437 | 46.67% |
| 428 | 446 | 46.67% |
| 428 | 448 | 63.33% |
| 428 | 504 | 66.67% |
| 428 | 544 | 63.33% |
| 428 | 551 | 63.33% |
| 429 | 437 | 43.33% |
| 429 | 446 | 40.00% |
| 429 | 448 | 53.33% |
| 429 | 504 | 53.33% |
| 429 | 544 | 70.00% |
| 429 | 551 | 56.67% |
| 437 | 446 | 56.67% |
| 437 | 448 | 60.00% |
| 437 | 504 | 56.67% |
| 437 | 544 | 50.00% |
| 437 | 551 | 56.67% |
| 446 | 448 | 63.33% |
| 446 | 504 | 56.67% |
| 446 | 544 | 56.67% |
| 446 | 551 | 46.67% |
| 448 | 504 | 56.67% |
| 448 | 544 | 56.67% |
| 448 | 551 | 70.00% |
| 504 | 544 | 73.33% |
| 504 | 551 | 53.33% |
| 544 | 551 | 53.33% |

As shown in Table 7, the genotypes of sample 218 and sample 403, sample 301 and sample 429, sample 304 and sample 446, sample 308 and sample 319, and sample 408 and sample 448 were completely consistent. These five sets of feces samples each were from one same individual. Sample 230 and sample 323 had the same genotype at each locus, and sample 406 had only one genotype difference from that of sample 230 and sample 323 separately. These three feces samples were determined to come from the same individual. There was only one genotype difference between sample 215 and sample 409, or sample 216 and sample 410. These two groups of samples were determined to come from the same individual according to the determination criteria.

In summary, it was one group (230-323-406) with 3 feces samples from the same individual. There were 7 groups (218-403, 301-429, 304-446, 308-319, 408-448, 215-409, and 216-410) with 2 feces samples from the same individual. The above results indicated that the 33 feces samples of Procapra przewalskii subadults were derived from 24 Procapra przewalskii individuals. According to the records of the rescue center, there were 9 new-born and surviving Procapra przewalskii in 2020, and there were 15 new-born and surviving Procapra przewalskii in 2021. That is to say, there were 24 subadults by April 2022, indicating that the molecular identification results were consistent with the actual situation. Therefore, the individual identification of Procapra przewalskii with the 15 SSR loci of the present disclosure showed reliable results.

The above descriptions are merely preferred embodiments of the present disclosure. It should be noted that those of ordinary skill in the art may further make several improvements and modifications without departing from the principle of the present disclosure, and such improvements and modifications should be deemed as falling within the claimed scope of the present disclosure.

1. A combination of single sequence repeat (SSR) molecular marker for Procapra przewalskii, the combination of molecular markers comprises one or more of PR-6, PR-7, PR-8, PR-10, PR-12, PR-14, PR-16, PR-22, PR-25, PR-26, PR-28, PR-30, PR-40, PR-42, PR-46, PR-53, PR-58, PR-63, PR-64, PR-65, PR-69, PR-71, PR-72, PR-85, PR-86, and PR-97; wherein
the SSR molecular markers are sequentially amplified by following primer pairs, the primer pairs have sequences set forth in SEQ ID NO: 1-2, SEQ ID NO: 3-4, SEQ ID NO: 5-6, SEQ ID NO: 7-8, SEQ ID NO: 9-10, SEQ ID NO: 11-12, SEQ ID NO: 13-14, SEQ ID NO: 15-16, SEQ ID NO: 17-18, SEQ ID NO: 19-20, SEQ ID NO: 21-22, SEQ ID NO: 23-24, SEQ ID NO: 25-26, SEQ ID NO: 27-28, SEQ ID NO: 29-30, SEQ ID NO: 31-32, SEQ ID NO: 33-34, SEQ ID NO: 35-36, SEQ ID NO: 37-38, SEQ ID NO: 39-40, SEQ ID NO: 41-42, SEQ NO: 43-44, SEQ ID NO: 45-46, SEQ ID NO: 47-48, SEQ ID NO: 49-50, SEQ ID NO: 51-52.

2. The combination of SSR molecular marker for Procapra przewalskii according to claim 1, the combination of molecular markers comprises the PR-6, the PR-8, the PR-12, the PR-22, the PR-25, the PR-30, the PR-40, the PR-58, the PR-63, the PR-69, the PR-71, the PR-72, the PR-85, the PR-86, and the PR-97.

3. (canceled)

4. A kit for identifying an individual of Procapra przewalskii, comprising a primer combination of SSR molecular marker for Procapra przewalskii, comprising any one or more of primer pairs, the primer pairs have sequences set forth in SEQ ID NO: 1-2, SEQ ID NO: 3-4, SEQ ID NO: 5-6, SEQ ID NO: 7-8, SEQ ID NO: 9-10, SEQ ID NO: 11-12, SEQ ID NO: 13-14, SEQ ID NO: 15-16, SEQ ID NO: 17-18, SEQ ID NO: 19-20, SEQ ID NO: 21-22, SEQ ID NO: 23-24, SEQ ID NO: 25-26, SEQ ID NO: 27-28, SEQ ID NO: 29-30, SEQ ID NO: 31-32, SEQ ID NO: 33-34, SEQ ID NO: 35-36, SEQ ID NO: 37-38, SEQ ID NO: 39-40, SEQ ID NO: 41-42, SEQ NO: 43-44, SEQ ID NO: 45-46, SEQ ID NO: 47-48, SEQ ID NO: 49-50, SEQ ID NO: 51-52.

5. The kit according to claim 4, further comprising a genome extraction reagent and a PCR reaction reagent.

6. The kit according to claim 4, further comprising a reagent for capillary electrophoresis.

7. (canceled)

8. A method for detection of a population genetic diversity of Procapra przewalskii, comprising using a primer combination of SSR molecular marker for Procapra przewalskii, comprising any one or more of primer pairs, the primer pairs have sequences set forth in SEQ ID NO: 1-2, SEQ ID NO: 3-4, SEQ ID NO: 5-6, SEQ ID NO: 7-8, SEQ ID NO: 9-10, SEQ ID NO: 11-12, SEQ ID NO: 13-14, SEQ ID NO: 15-16, SEQ ID NO: 17-18, SEQ ID NO: 19-20, SEQ ID NO: 21-22, SEQ ID NO: 23-24, SEQ ID NO: 25-26, SEQ ID NO: 27-28, SEQ ID NO: 29-30, SEQ ID NO: 31-32, SEQ ID NO: 33-34, SEQ ID NO: 35-36, SEQ ID NO: 37-38, SEQ ID NO: 39-40, SEQ ID NO: 41-42, SEQ NO: 43-44, SEQ ID NO: 45-46, SEQ ID NO: 47-48, SEQ ID NO: 49-50, SEQ ID NO: 51-52.

9-10. (canceled)

11. The method according to claim 8, wherein the primer combination of SSR molecular marker for Procapra przewalskii is in the kit according to claim 4.
